(19) United States
(12) Patent Application Publication
(10) Pub. No.: US 2003/0068787 A1
(43) Pub. Date: Apr. 10, 2003

(54) Antibody specifically binding cyclophilin-type peptidyl-prolyl cis/trans isomerase

(75) Inventors: Jennifer L. Jackson, Fremont, CA (US); Neil C. Corley, Castro Valley, CA (US); Karl J. Guegler, Menlo Park, CA (US); Chandra S. Arvizu, San Jose, CA (US)

Correspondence Address: INCYTE GENOMICS, INC., 3160 PORTER DRIVE, PALO ALTO, CA, 94304, US

(73) Assignee: Incyte Genomics, Inc., Palo Alto, CA

(21) Appl. No.: 10/177,900

(22) Filed: Jun. 20, 2002

Related U.S. Application Data
(63) Continuation-in-part of application No. 09/440,828, filed on Nov. 15, 1999, now Pat. No. 6,458,575.
Application No. 09/440,828 is a division of application No. 09/136,442, filed on Aug. 19, 1998, now Pat. No. 6,030,825.

(51) Int. Cl. C12P 21/04; C12N 9/00; C07K 16/00

(52) U.S. Cl. 435/70.21; 530/388.1; 435/183; 530/413

(57) ABSTRACT

The invention provides a human cyclophilin-type peptidyl-prolyl cis/trans isomerase (CPCI), a cDNA that encodes CPCI and an antibody that specifically binds CPCI. The invention also provides methods to diagnose, to stage, to treat, or to monitor the treatment of disorders associated with expression of CPCI.

This application is a continuation-in-part of U.S. Ser. No. 09/440,828, filed Nov. 15, 1999, which is a divisional of U.S. Pat. No. 6,030,825, issued Feb. 29, 2000, which matured from U.S. Ser. No. 09/136,442, filed Aug. 19, 1998.

### FIELD OF THE INVENTION

This invention relates to a cyclophilin-type peptidyl-prolyl cis/trans isomerase, a cDNA encoding the enzyme, and to an antibody that specifically binds the enzyme and to the use of the these compositions to diagnose, to stage, to treat, or to monitor the progression or treatment of neoplastic disorders and immune response.

### BACKGROUND OF THE INVENTION

Numerous biochemical reactions involve the isomerization of a substrate. Enzymes which catalyze such reactions are known as isomerases. A number of isomerases have been described which catalyze steps in a wide variety of biochemical pathways. These pathways include protein folding, phototransduction, and various anabolic and catabolic processes in organisms ranging from bacteria to human.

One class of isomerases is known as peptidyl-prolyl cis-trans isomerases (PPIases). PPIases catalyze the cis to trans isomerization of certain proline imidic bonds in proteins. Two families of PPIases are the FK506 binding proteins (FKBPs) and cyclophilins (CyPs). FKBPs bind potent immunosuppressants thereby inhibiting signaling pathways in T-cells. Specifically, the PPIase activity of FKBPs is inhibited by binding of FK506 or rapamycin. There are five members of the FKBP family which are named according to their calculated molecular masses (FKBP12, FKBP13, FKBP25, FKBP52, and FKBP65), and localized to different regions of the cell where they associate with different protein complexes (Coss et al. (1995) J Biol Chem 270:29336-29341; Schreiber (1991) Science 251:283-287).

CyP was originally characterized as the receptor for the immunosuppressant drug cyclosporin, an inhibitor of T-cell activation. Thus, the peptidyl-prolyl isomerase activity of CyP may be part of the signaling pathway that leads to T-cell activation. Subsequent work demonstrated that CyP is active in correct protein folding and/or protein trafficking and may be involved in assembly/disassembly of protein complexes and regulation of protein activity. For example, in Drosophila, the CyP NinaA is required for correct localization of rhodopsins, while a mammalian CyP (Cyp40) is part of the Hsp90/Hsc70 complex that binds steroid receptors. The mammalian CypA has been shown to bind the gag protein from human immunodeficiency virus 1 (HIV-1), an interaction that can be inhibited by cyclosporin. Since cyclosporin has potent anti-HIV-1 activity, CypA may also function in HIV-1 replication. Finally, Cyp40 has been shown to bind and inactivate the transcription factor c-Myb, an effect that is reversed by cyclosporin. This effect implicates CyPs in the regulation of transcription, transformation, and differentiation (Bergsma et al. (1991) J Biol Chem 266:23204-23214; Hunter (1998) Cell 92:141-143; and Leverson and Ness (1998) Mol Cell 1:203-211).

The discovery of a new cyclophilin-type peptidyl-prolyl cis/trans isomerase, a cDNA encoding the enzyme, and to an antibody that specifically binds the enzyme satisfies a need in the art by providing new compositions which are useful to diagnose, to stage, to treat and to monitor the treatment of neoplastic disorders and immune response.

### SUMMARY OF THE INVENTION

The invention is based on the discovery of a new human cyclophilin-type peptidyl-prolyl cis/trans isomerase, a cDNA encoding the enzyme, and to an antibody that specifically binds the enzyme. These compositions may be used to diagnose, to stage, to treat, or to monitor the progression or treatment of neoplastic disorders and immune response.

The invention provides an isolated cDNA comprising a nucleic acid sequence encoding a protein having the amino acid sequence of SEQ ID NO:1. The invention also provides an isolated cDNA or the complement thereof selected from a nucleic acid sequence of SEQ ID NO:2; a fragment of SEQ ID NO:2 selected from SEQ ID NOs:3-5; an oligonucleotide extending from about A615 to about nucleotide A675 of SEQ ID NO:2; and mammalian homologs of SEQ ID NO:2 selected from SEQ ID NOs:6-10.

The invention provides a vector containing the cDNA encoding CPCI, a host cell containing the vector and a method for using the cDNA to make the protein, the method comprising culturing the host cell containing the vector containing the cDNA encoding the protein under conditions for expression and recovering the protein from the host cell culture. The invention also provides a transgenic cell line or organism comprising the vector containing the cDNA encoding CPCI. The invention further provides a composition, a substrate or a probe comprising the cDNA, a fragment, a variant, or complements thereof, which can be used in methods of detection, screening, and purification. In one aspect, the probe is a single-stranded complementary RNA or DNA molecule.

The invention provides a method for using a cDNA to detect the differential expression of a nucleic acid in a sample comprising hybridizing a probe to the nucleic acids, thereby forming hybridization complexes and comparing hybridization complex formation with a standard, wherein the comparison indicates the differential expression of the cDNA in the sample. In one aspect, the method of detection further comprises amplifying the nucleic acids of the sample prior to hybridization. In another aspect, the method showing differential expression of the cDNA is used to diagnose a cancer or complications thereof.

The invention provides a method for using a cDNA to screen a library or plurality of molecules or compounds to identify at least one ligand which specifically binds the cDNA, the method comprising combining the cDNA with the molecules or compounds under conditions to allow specific binding and detecting specific binding to the cDNA, thereby identifying a ligand which specifically binds the cDNA. In one aspect, the molecules or compounds are selected from antisense molecules, artificial chromosome constructions, branched nucleic acids, DNA molecules, enhancers, peptides, peptide nucleic acids, proteins, RNA molecules, repressors, and transcription factors.

The invention provides a method for using a cDNA to purify a ligand which specifically binds the cDNA, the method comprising attaching the cDNA to a substrate, contacting the cDNA with a sample under conditions to allow specific binding, and dissociating the ligand from the cDNA, thereby obtaining purified ligand.

The invention provides a purified protein or a portion thereof selected from the group consisting of an amino acid sequence of SEQ ID NO:1, a variant having at least 85% identity to the amino acid sequence of SEQ ID NO:1, an antigenic determinant or biologically active portion of SEQ ID NO:1 from about V3 to about A15, from about R84 to about L12, and from about G85 to about Q100 of SEQ ID NO: 1. The invention also provides a composition comprising the purified protein and a pharmaceutical carrier. A method for diagnosing a neoplastic disorder or immune response comprising performing an assay to quantify the amount of the protein of claim 1 expressed in a sample and comparing the amount of protein expressed to standards, thereby diagnosing a neoplastic disorder or immune response. In one aspect, the disorder is squamous cell carcinoma or atherosclerosis. In a second aspect, the assay is selected from two-dimensional polyacrylamide gel electrophoresis, western analysis, mass spectrophotometry, enzyme-linked immunosorbent assays, radioimmunoassays, fluorescence-activated cell sorting, protein arrays, and antibody arrays.

The invention provides a method for using a protein to screen a library or a plurality of molecules or compounds to identify at least one ligand, the method comprising combining the protein with the molecules or compounds under conditions to allow specific binding and detecting specific binding, thereby identifying a ligand which specifically binds the protein. In one aspect, the molecules or compounds are selected from agonists, antagonists, antibodies, DNA molecules, small drug molecules, immunoglobulins, inhibitors, mimetics, peptides, peptide nucleic acids, proteins, and RNA molecules. In another aspect, the ligand is used to treat a subject with a cancer or complications thereof. The invention also provides an agonist which specifically binds the protein having the amino acid sequence of SEQ ID NO:1. The invention further provides an antagonist which specifically binds the protein having the amino acid sequence of SEQ ID NO:1. The invention still further provides a small drug molecule which specifically binds the protein having the amino acid sequence of SEQ ID NO:1. The invention yet further provides a therapeutic antibody which specifically binds the protein having the amino acid sequence of SEQ ID NO:1.

The invention provides a method for using a protein to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein. In one aspect, the antibody is selected from an intact immunoglobulin molecule, a polyclonal antibody, a monoclonal antibody, a chimeric antibody, a recombinant antibody, a bispecific antibody, a multispecific antibody, a humanized antibody, a single chain antibody, a Fab fragment, an F(ab′)₂ fragment, an Fv fragment; an agonist antibody, and an antibody-peptide fusion protein.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides purified antibodies which bind specifically to a protein. The invention also provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the amount of complex formation when compared to standards is diagnostic of cancer or complications of cancer and in particular squamous cell carcinoma.

The invention provides a method for immunopurification of a protein comprising attaching an antibody to a substrate, exposing the antibody to a sample containing protein under conditions to allow antibody:protein complexes to form, dissociating the protein from the complex, and collecting purified protein. The invention also provides a kit, a substrate or an array upon which CPCI or an antibody which specifically binds CPCI are immobilized.

The invention provides a method for inserting a heterologous marker gene into the genomic DNA of a mammal to disrupt the expression of the endogenous polynucleotide. The invention also provides a method for using a cDNA to produce a mammalian model system, the method comprising constructing a vector containing the cDNA selected from SEQ ID NOs:2-10, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, mammalian model system.

### BRIEF DESCRIPTION OF THE FIGURES AND TABLE

FIGS. 1A-1C show the amino acid sequence (SEQ ID NO:1) and nucleic acid sequence (SEQ ID NO:2) of CPCI. The alignment was produced using MACDNASIS PRO software (Hitachi Software Engineering, S. San Francisco Calif.).

FIGS. 2A and 2B show the amino acid sequence alignment between CPCI (2925455CD1; SEQ ID NO:1), C. elegans cyclophilin isoform 10 (GI 1155225; SEQ ID NO:11), and human cyclophilin-like protein (GI 1199600; SEQ ID NO: 12), produced using the MEGALIGN program of LASERGENE software (DNASTAR Madison Wis.).

Table 1 shows the programs, their descriptions, references, and threshold parameters used to analyze CPCI.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Antibody” refers to an intact immunoglobulin molecule, a polyclonal antibody, a monoclonal antibody, a chimeric antibody, a recombinant antibody, a bispecific antibody, a multispecific antibody, a humanized antibody, a single chain antibody, a Fab fragment, an F(ab′)₂ fragment, an Fv fragment; an agonist antibody, and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

The “complement” of a cDNA of the Sequence Listing refers to a nucleic acid molecule which is completely complementary over its full length and which will hybridize to a nucleic acid molecule under conditions of high stringency.

“CDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to a polynucleotide and a labeling moiety; a purified protein and a pharmaceutical carrier or a heterologous, labeling or purification moiety; an antibody and a labeling moiety or pharmaceutical agent; and the like.

“CPCI” refers to the amino acid sequences of purified CPCI obtained from any species, particularly a mammalian species, including bovine, ovine, porcine, murine, equine, and preferably the human species, from any source, whether natural, synthetic, semi-synthetic, or recombinant.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to neoplastic or immune conditions, diseases or syndromes in which CPCI or the mRNA encoding CPCI are differentially expressed; these include cancers, and in particular squamous cell carcinoma, and immune responses, and in particular, atherosclerosis.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses two-dimensional polyacrylamide electrophoresis (2D-PAGE, western analysis, mass spectrophotometry (MS), enzyme-linked immunosorbent assays (ELISAs), radioimmunoassays (RIAs), and fluorescence activated cell sorting (FACS) or arrays and labeling moieties or antibodies to detect expression in a sample. The nucleic acids, proteins, or antibodies may be used in solution or attached to a substrate, and their detection is based on methods and labeling moieties well known in the art.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-.G-5′. Hybridization conditions, degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997, supra). BLAST2 may be used in a standardized and reproducible way to insert gaps in one of the sequences in order to optimize alignment and to achieve a more meaningful comparison between them. “Similarity” uses the same algorithms but takes conservative substitution of residues into account. In proteins, similarity exceeds identity in that substitution of a valine for a leucine or isoleucine, is counted in calculating the reported percentage. Substitutions which are considered to be conservative are well known in the art.

“Immune response” can refer to conditions associated with inflammation, trauma, immune disorders, infectious disease or complications associated with cancer. These conditions are characterized by expression of various factors, e.g., cytokines, chemokines, hormones, and other signaling molecules which may affect cellular and systemic defense systems.

“Isolated or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels and dyes include but are not limited to anthocyanins, β glucuronidase, BIODIPY, Coomassie blue, Cy3 and Cy5, DAPI, digoxigenin, fluorescein, FITC, gold, green fluorescent protein, lissamine, luciferase, phycoerythrin, rhodamine, spyro red, silver, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplicon, amplimer, primer, and oligomer.

A “pharmaceutical agent” may be an antisense molecule, a protein, a small drug molecule, a radionuclide, a cytotoxin such as vincristine, vinblastine, cisplatin, doxorubicin, methotrexate, and the like.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using algorithms of LASERGENE software (DNASTAR, Madison Wis.) or MACDNASIS PRO software (Hitachi Software Engineering). An “oligopeptide” is an amino acid sequence from about five residues to about 15 residues that is used as part of a fusion protein to produce an antibody.

“Sample” is used in its broadest sense as containing nucleic acids, proteins, and antibodies. A sample may comprise a bodily fluid such as ascites, blood, cerebrospinal fluid, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like.

“Specific binding” refers to a precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The invention is based on the discovery of a new human cyclophilin-type peptidyl-prolyl cis/trans isomerase (CPCI), the cDNAs encoding CPCI, and an antibody which specifically binds CPCI and on the use of these compositions to diagnose, to stage, to treat, or to monitor the progression or treatment of neoplastic disorders and immune response.

Nucleic acids encoding the CPCI of the present invention were identified in Incyte Clone 2925455 from the SININOT04 cDNA library using a BLAST search for amino acid sequence alignments. A consensus sequence, SEQ ID NO:2, was derived from the following overlapping and/or extended nucleic acid sequences: Incyte Clones 2925455H1 (SININOT04), 2619977R6 (KERANOT02), and 586886F1 (UTRSNOT01). A fragment of SEQ ID NO:2 from about nucleotide A615 to about nucleotide A675 is useful as a hybridization probe.

Northern analysis showed the expression of this sequence in various libraries, at least 55% of which are associated with cancer or proliferating tissue and at least 32% of which are associated with the immune response. Of particular note is the expression of CPCI in tumors of lung, prostate, ovary and breast. Transcript imaging as detailed in EXAMPLE V shows the differential expression of CPCI in squamous cell carcinoma. In EXAMPLE VI, microarray data shows the differential expression of CPCI in activated macrophages following treatment with LPS wherein differential expression mimics immune response in a model system for atherosclerosis.

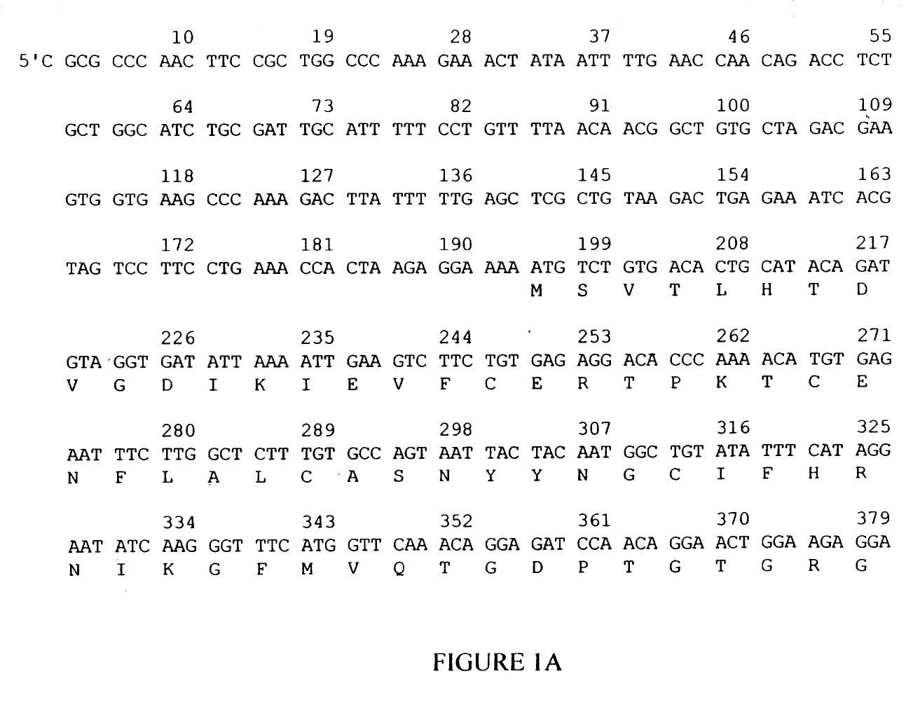

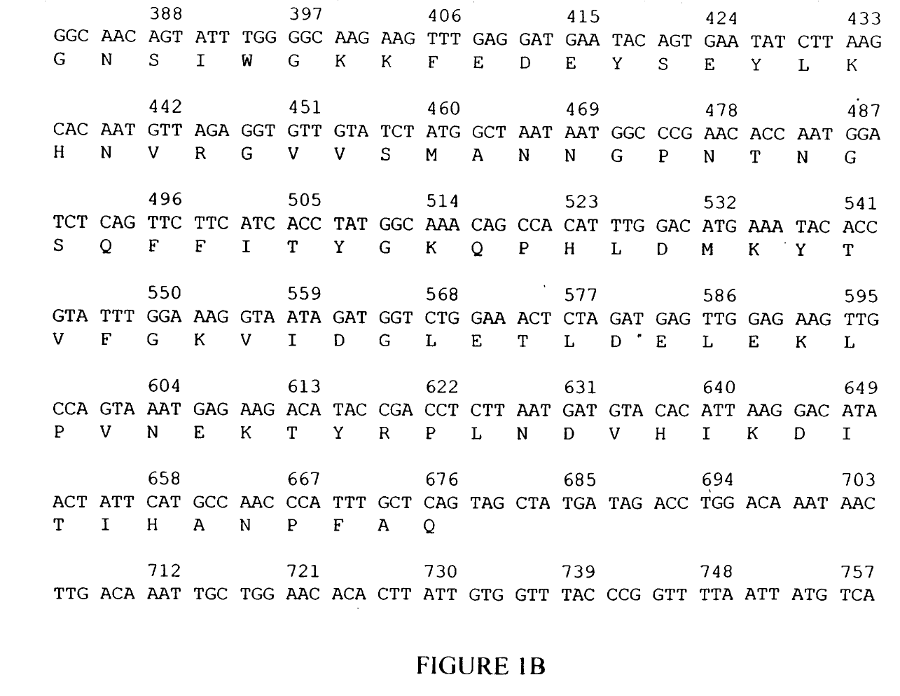

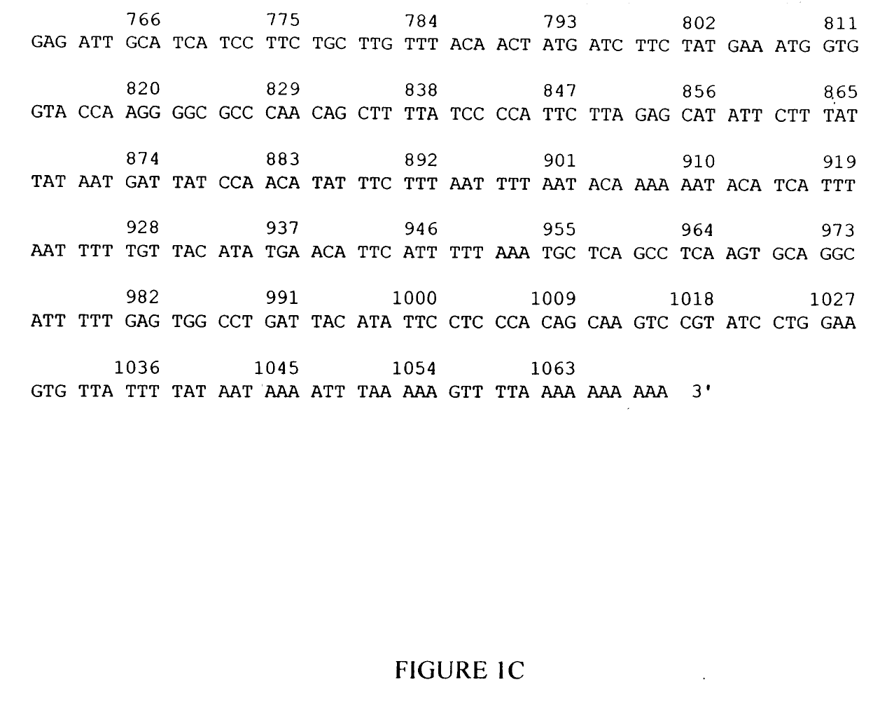

In one embodiment, the invention encompasses a protein comprising a polypeptide having the amino acid sequence of SEQ ID NO:1 as shown in FIGS. 1A-1C. CPCI is 161 amino acids in length and has a potential N-glycosylation site at residue N97. In addition, CPCI has a potential casein kinase II phosphorylation site at residue T127, three potential protein kinase C phosphorylation sites at residues T21, T59, and T140, and two potential tyrosine kinase phosphorylation sites at residues Y75 and Y78. HMM analysis using the PFAM database identified CPCI as a peptidyl-prolyl cis/trans isomerase, with the region from amino acids V3 through A156 receiving a score of 192 bits. BLIMPS analysis using the BLOCKS database identified CPCI as a cyclophilin-type peptidyl-prolyl cis/trans isomerase (BL00170), which the algorithm characterizes using three segments, BL00170A, BL00170B, and BL00170C. The region of CPCI from R84 through L128, corresponding to segment BL00170C, received a score of 1476 on a strength of 1662, and was supported by the presence of BL00170A and BL00170B with a P value less than 4.6×10⁻⁶. BLIMPS analysis using the PRINTS database also identified CPCI as a cyclophilin-type peptidyl-prolyl cis/trans isomerase (PR00153), which the algorithm characterizes using five segments, PR00153A, PR00153B, PR00153C, PR00153D, and PR00153E. The region of CPCI from residue G85 through residue Q100, corresponding to PR00153C, received a score of 1450 on a strength of 1339, and was supported by the presence of PR00153A, PR00153B, PR00153D, and PR00153E with a P value less of than 4.6×10⁻¹⁶. The portions of SEQ ID NO:1 from about V3 to about A15, from about R84 to about L12, and from about G85 to about Q100 may be used as antigenic determinants or to screen for antibodies which specifically bind CPCI. These antibodies may be used-to elucidate the differential expression of CPCI and to diagnose a neoplastic disorders such as squamous cell carcinoma or an immune response such as atherosclerosis.

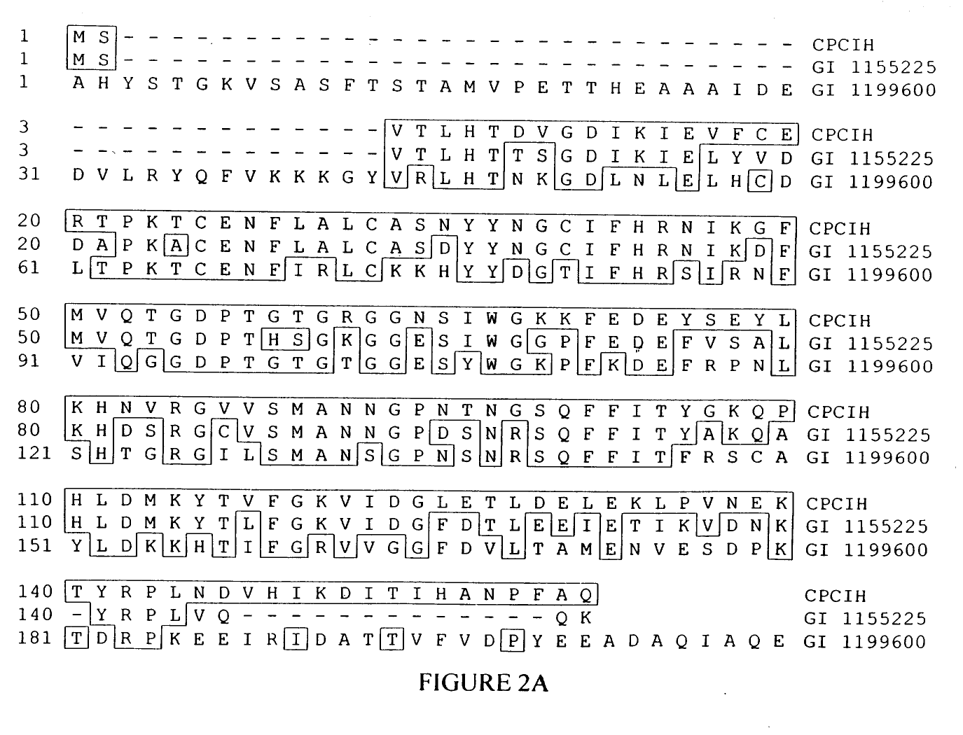

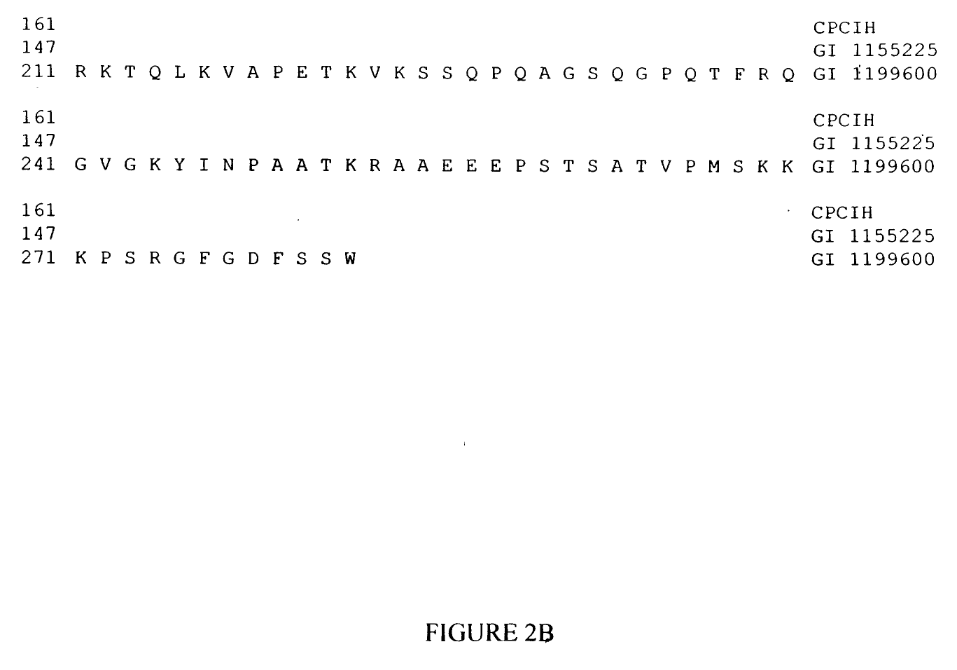

As shown in FIGS. 2A and 2B, CPCI has chemical and structural similarity with cyclophilin isoform 10 from C. elegans (GI 1155225; SEQ ID NO:11), and cyclophilin-like protein from H. sapiens (GI 1199600; SEQ ID NO:12). In particular, CPCI and cyclophilin isoform 10 share 65% identity, while CPCI and cyclophilin-like protein share 49% identity.

Mammalian homologs of the cDNA encoding CPCI were identified using BLAST2 with default parameters and the ZOOSEQ databases (Incyte Genomics, Palo Alto Calif.). These mammalian cDNAs have at least 90% identity to all or part of the coding region of the human cDNA as shown in the table below. The first column represents the SEQ ID NO: for the mammalian cDNAs; the second column, the Incyte ID for the mammalian cDNA; the third column, the species; the fourth column, the percent identity to the human cDNA; and the fifth column, the nucleotide alignment of the mammalian to the human cDNA of SEQ ID NO:2.

| SEQ ID_{Mam} | Incyte ID_{Mam} | Species | Identity | Nt_{Human} Alignment |
|---|---|---|---|---|
| 6 | 222746_Rn.1 | Rat | 92% | 193-694 |
| 7 | 252329_Rn.1 | Rat | 99% | 118-265 |
| 8 | 023828_Cf.1 | Dog | 96% | 184-365 |
| 9 | 024646_Cf.1 | Dog | 91% | 545-798 |
| 10 | 023075_Mm.3 | Mouse | 93% | 191-697 |

The cDNAs of SEQ ID NOs:2-10 may be used in hybridization, amplification, and screening technologies to identify and distinguish among SEQ ID NO:2 and related molecules. The mammalian cDNAs, SEQ ID NOs:6-10, may be used to produce transgenic cell lines or organisms which are model systems for human disorders and upon which the toxicity and efficacy of therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

Characterization and Use of the Invention

It will be appreciated by those skilled in the art that as a result of the degeneracy of the genetic code, a multitude of cDNAs encoding CPCI, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring CPCI, and all such variations are to be considered as being specifically disclosed.

cDNA Libraries

In a particular embodiment disclosed herein, mRNA is isolated from cells and tissues using methods which are well known to those skilled in the art and used to prepare the cDNA libraries. The Incyte clones were isolated from cDNA libraries prepared as described in the EXAMPLES. The consensus sequences are chemically and/or electronically assembled from sequence fragments including Incyte cDNAs and extension and/or shotgun sequences using computer programs such as PHRAP (P Green, University of Washington, Seattle Wash.), and the AUTOASSEMBLER application (ABI). After verification of the 5′ and 3′ sequence, at least one of the representative cDNAs which encodes CPCI is designated a reagent. In this case, the reagent cDNA is 2619977CA2, a verified extension of Incyte clones 2619977H1 and 2619977R6 from the KERANOT02 cDNA library. The reagent cDNA was used in the construction of the microarray used in EXAMPLE VI.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Biosciences (APB), Piscataway N.J.), or combinations of polymerases and proofreading exonucleases (Invitrogen, Carlsbad Calif.). Sequence preparation is automated with machines such as the MICROLAB 2200 system (Hamilton, Reno Nev.) and the DNA ENGINE thermal cycler (M J Research, Watertown Mass.) and sequencing, with the PRISM 3700, 377 or 373 DNA sequencing systems (ABI) or the MEGABACE 1000 DNA sequencing system (APB).

The nucleic acid sequences of the cDNAs presented in the Sequence Listing were prepared by automated methods and may contain occasional sequencing errors and unidentified nucleotides (N) that reflect state-of-the-art technology at the time the cDNA was sequenced. Occasional sequencing errors and Ns may be resolved, and SNPs verified, either by resequencing the cDNA or using algorithms to compare multiple sequences from EST and full length sequence databases; both of these techniques are well known to those skilled in the art who wish to practice the invention. The sequences may be analyzed using a variety of algorithms described in Ausubel et al. (1997; Short Protocols in Molecular Biology, John Wiley & Sons, New York N.Y., unit 7.7) and in Meyers (1995; Molecular Biology and Biotechnology, Wiley V C H, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, can be masked or removed to complete the transcribed sequence.

Extension of a Nucleic Acid Sequence

The sequences of the invention may be extended using various PCR-based methods known in the art. For example, the XL-PCR kit (ABI), nested primers, and cDNA or genomic DNA libraries may be used to extend the nucleic acid sequence. For all PCR-based methods, primers may be designed using software, such as OLIGO primer analysis software (Molecular Biology Insights, Cascade Colo.) to be about 22 to 30 nucleotides in length, to have a GC content of about 50% or more, and to anneal to a target molecule at temperatures from about 55C to about 68C. When extending a sequence to recover regulatory elements, genomic, rather than cDNA libraries are used.

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the CPCI, allelic variants, or related molecules. The probe may be DNA or RNA, may be single-stranded, and should have at least 50% sequence identity to any of the nucleic acid sequences, SEQ ID NOs:2-9. Hybridization probes may be produced using oligolabeling, nick-translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using kits such as those provided by APB.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5× SSC with 1% sodium dodecyl sulfate (SDS) at 60C, which permits the formation of a hybridization complex between nucleic acid sequences that contain some mismatches. Subsequent washes are performed at higher stringency with buffers such as 0.2× SSC with 0.1% SDS at either 45C (medium stringency) or 68C (high stringency). At high stringency, hybridization complexes will remain stable only where the nucleic acids are completely complementary. In some membrane-based hybridizations, from about 35% to about 50% formamide can be added to the hybridization solution to reduce the temperature at which hybridization is performed. Background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and-single nucleotide polymorphisms. Arrays may be used to determine gene function; to understand the genetic basis of a condition, disease, or disorder; to diagnose a condition, disease, or disorder; and to develop and monitor the activities of pharmaceutical agents. (See, e.g., U.S. Pat. No. 5,474,796; Schena et al. (1996) Proc Natl Acad Sci 93:10614-10619; Heller et al. (1997) Proc Natl Acad Sci 94:2150-2155; U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes, yeast artificial chromosomes, bacterial artificial chromosomes, bacterial P1 constructions, or the cDNAs of libraries made from single chromosomes.

QPCR

QPCR is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system (ABI) which consists of a 96-well thermal cycler connected to a laser and charge-coupled device (CCD) optics system. To perform QPCR, a PCR reaction is carried out in the presence of a doubly labeled probe. The probe, which is designed to anneal between the standard forward and reverse PCR primers, is labeled at the 5′ end by a flourogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid, the sooner an increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T} values, preparing a standard curve, and determining starting copy number is performed using SEQUENCE DETECTOR 1.7 software (ABI).

Expression

Any one of a multitude of cDNAs encoding CPCI may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors or plant cell systems transformed with expression vectors containing viral and/or bacterial elements (Ausubel supra, unit 16). In mammalian cell systems, an adenovirus transcription/translation complex may be utilized. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6× His, FLAG, MYC, and the like. GST and 6-His are purified using affinity matrices such as immobilized glutathione and metal-chelate resins, respectively. FLAG and MYC are purified using monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16).

Protein Identification

Several techniques have been developed which permit rapid identification of proteins using high performance liquid chromatography and mass spectrometry (MS). Beginning with a sample containing proteins, the method is: 1) proteins are separated using two-dimensional gel electrophoresis (2-DE), 2) selected proteins are excised from the gel and digested with a protease to produce a set of peptides; and 3) the peptides are subjected to mass spectral analysis to derive peptide ion mass and spectral pattern information. The MS information is used to identify the protein by comparing it with information in a protein database (Shevenko et al. (1996) Proc Natl Acad Sci 93:14440-14445).

Proteins are separated by 2DE employing isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Probes, Eugene Oreg.) that is compatible with MS. Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a MALDI-TOF (Matrix Assisted Laser Desorption/Ionization-Time of Flight), ESI (Electrospray Ionization), and TOF-TOF (Time of Flight/Time of Flight) machines are used to determine a set of highly accurate peptide masses. Using analytical programs, such as TURBOSEQUEST software (Finnigan, San Jose Calif.), the MS data is compared against a database of theoretical MS data derived from known or predicted proteins. A minimum match of three peptide masses is used for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation (CID) to produce an ion series. The resulting fragmentation ions are analyzed in a second round of MS, and their spectral pattern may be used to determine a short stretch of amino acid sequence (Dancik et al. (1999) J Comput Biol 6:327-342). Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf. Creighton (1984) Proteins, Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative high performance liquid chromatography and its composition confirmed by amino acid analysis or by sequencing (Creighton (1984) Proteins, Structures and Molecular Properties, W H Freeman, New York N.Y.).

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich), and dinitrophenol may be used to increase immunological response. In humans, BCG (bacilli Calmette-Guerin) and Corynebacterium parvum increase response. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopeptides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al. (1975) Nature 256:495-97; Kozbor et al. (1985) J Immunol Methods 81:31-42; Cote et al. (1983) Proc Natl Acad Sci 80:2026-2030; and Cole et al. (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are commonly used in immunoassays in which the protein-antibody complex must withstand rigorous manipulations. Low-affinity antibody preparations with Kₐ ranging from about 10⁶ to 10⁷ L/mole are used in immunopurification and similar procedures which ultimately require dissociation of the protein, in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are discussed in Catty (supra) and Ausubel (supra) pp. 11.1-11.31.

Diagnostics

Differential expression of CPCI as detected using the cDNA encoding CPCI or an antibody that specifically binds CPCI and any of the assays described herein can be used to diagnose a neoplastic disorder such as squamous cell carcinoma or an immune response such as atherosclerosis that is characterized by expression of CPCI.

Labeling of Molecules for Assay

A wide variety of reporter molecules and conjugation techniques are known by those skilled in the art and may be used in various nucleic acid, amino acid, and antibody assays. Synthesis of labeled molecules may be achieved using kits such as those supplied by Promega (Madison Wis.) or APB for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes).

Nucleic Acid Assays

The cDNAs, fragments, oligonucleotides, complementary RNA and nucleic acid molecules, and peptide nucleic acids may be used to detect and quantify differential gene expression for diagnosis of a disorder. Disorders associated with such differential expression of CPCI particularly include squamous cell carcinoma and atherosclerosis. The diagnostic assay may use hybridization or amplification technology to compare gene expression in a biological sample from a patient to standard samples in order to detect differential gene expression. Qualitative or quantitative methods for this comparison are well known in the art.

Expression Profiles

A gene expression profile comprises the expression of a plurality of cDNAs as measured by after hybridization with a sample. The cDNAs of the invention may be used as elements on a array to produce a gene expression profile. In one embodiment, the array is used to diagnose or monitor the progression of disease. Researchers can assess and catalog the differences in gene expression between healthy and diseased tissues or cells.

For example, the cDNA or probe may be labeled by standard methods and added to a biological sample from a patient under conditions for the formation of hybridization complexes. After incubation, the sample is washed and the amount of label (or signal) associated with hybridization complexes, is quantified and compared with a standard value. If complex formation in the patient sample is altered toward the disease standard, then expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose or stage that disorder.

By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disease, or disorder; or treatment of the condition, disease, or disorder. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Immunological methods for detecting and measuring complex formation as a measure of protein expression using either specific polyclonal or monoclonal antibodies are well known in the art. Examples of such techniques include western analysis, ELISAs, RIAs, FACS and protein and antibody arrays. Such immunoassays typically involve the measurement of complex formation between the protein and its specific antibody. These assays and their quantitation against purified, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. (See de Wildt et al. (2000) Nature Biotechnol 18:989-94.)

Therapeutics

Chemical and structural similarity exists between regions of CPCI and cyclophilin-type peptidyl-prolyl cis/trans isomerases, cyclophilin isoform 10 from C. elegans (GI 1155225; SEQ ID NO:11), and cyclophilin-like protein from H. sapiens (GI 1199600; SEQ ID NO:12). In addition, the expression of CPCI is closely associated with neoplastic disorders and immune response. In the treatment of disorders associated with increased CPCI activity, it is desirable to decrease the expression or activity of CPCI. In the treatment of disorders which are associated with decreased CPCI activity, it is desirable to provide the protein or to increase transcript expression.

In one embodiment, when decreased expression or activity of the protein is desired, an inhibitor, antagonist, antibody, or a pharmaceutical agent containing one or more of these molecules may be delivered. Such delivery may be effected by methods well known in the art and may include delivery by an antibody specifically targeted to the protein. Neutralizing antibodies which inhibit dimer formation are preferred for therapeutic use.

In one embodiment, CPCI may be administered to a subject to treat a disorder associated with decreased expression or activity of CPCI. Examples of such disorders include a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, anemia, asthma, atherosclerosis, bronchitis, cholecystitis, contact dermatitis, Crohn's disease, atopic dermatitis, diabetes mellitus, emphysema, atrophic gastritis, hypereosinophilia, irritable bowel syndrome, multiple sclerosis, myasthenia gravis, osteoarthritis, osteoporosis, pancreatitis, psoriasis, rheumatoid arthritis, scleroderma, systemic lupus erythematosus, systemic sclerosis, ulcerative colitis, and complications of cancer, hemodialysis, and extracorporeal circulation, viral, bacterial, fungal, parasitic, protozoal, and helminthic infections, and trauma.; or a reproductive disorder such endometriosis, polycystic ovary syndrome, fibrocystic breast disease, galactorrhea; benign prostatic hyperplasia, and prostatitis.

In another embodiment, when increased expression or activity of the protein is desired, the protein, an agonist, an enhancer, or a pharmaceutical agent containing one or more of these molecules may be delivered. Such delivery may be effected by methods well known in the art and may include delivery of a pharmaceutical agent by an antibody specifically targeted to the protein.

Any of the cDNAs, complementary molecules, or fragments thereof, proteins or portions thereof, vectors delivering these nucleic acid molecules or expressing the proteins, and their ligands may be administered in combination with other pharmaceutical agents. Selection of the agents for use in combination therapy may be made by one of ordinary skill in the art according to conventional pharmaceutical principles. A combination of pharmaceutical agents may act synergistically to affect treatment of a particular disorder at a lower dosage of each agent.

Modification of Gene Expression Using Nucleic Acids

Gene expression may be modified by designing complementary or antisense molecules (DNA, RNA, or peptide nucleic acid) to the control, 5′, 3′, or other regulatory regions of the gene encoding CPCI. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of peptide nucleic acids and can be extended to other nucleic acid molecules. Either the inclusion of nontraditional bases such as inosine, queosine, and wybutosine, or the modification of adenine, cytidine, guanine, thymine, and uridine with acetyl-, methyl-, thio-groups renders the molecule more resistant to endogenous endonucleases.

cDNA Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

Screening and Purification Assays

The cDNA encoding CPCI may be used to screen a library or a plurality of molecules or compounds for specific binding affinity. The libraries may be antisense molecules, artificial chromosome constructions, branched nucleic acid molecules, DNA molecules, peptides, peptide nucleic acid, proteins such as transcription factors, enhancers, or repressors, RNA molecules, ribozymes, and other ligands which regulate the activity, replication, transcription, or translation of the endogenous gene. The assay involves combining the cDNA with a library or plurality of molecules or compounds under conditions allowing specific binding, and detecting specific binding to identify at least one molecule which specifically binds the cDNA.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein to purify a ligand would involve combining the protein with a sample under conditions to allow specific binding, detecting specific binding between the protein and ligand, recovering the bound protein, and using a chaotropic agent to separate the protein from the purified ligand.

In an additional embodiment, CPCI may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify agonists, antagonists, antibodies, DNA molecules, small drug molecules, immunoglobulins, inhibitors, mimetics, peptides, peptide nucleic acids, proteins, and RNA molecules or any other ligand, which specifically binds the protein.

In one aspect, this invention contemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity or therapeutic potential.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, gestation period, numbers of progeny, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to test whether long term administration may elicit toxicity, teratogenesis, or carcinogenesis. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies. Embryonic Stem Cells Embryonic (ES) stem cells isolated from rodent embryos retain the ability to form embryonic tissues. When ES cells are placed inside a carrier embryo, they resume normal development and contribute to tissues of the live-born animal. ES cells are used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularisand Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I. Construction of cDNA Libraries

The SININOT04 cDNA library was constructed using RNA isolated from the microscopically normal ileum obtained from a 26-year-old Caucasian male during a partial colectomy, permanent colostomy, and an incidental appendectomy. Pathology indicated moderately to severely active Crohn's disease, involving a central segment of terminal ileum, cecum, and ascending colon. The specimen showed transmural inflammation with skip areas, mural fibrosis, fissuring ulceration, and lymphoid aggregates present in all layers of the bowel wall.

The frozen tissue was homogenized and lysed in TRIZOL reagent (1 gm tissue/10 ml reagent; Invitrogen) using a Polytron homogenizer (PT-3000; Brinkmann Instruments, Westbury N.Y.). After a brief incubation on ice, chloroform was added (1:5 v/v), and the lysate was centrifuged. The upper chloroform layer was removed to a fresh tube, and the RNA extracted with isopropanol, resuspended in DEPC-treated water, and treated with DNAse for 25 min at 37° C. The RNA was re-extracted twice with acid phenol-chloroform, pH 4.7, and precipitated using 0.3M sodium acetate and 2.5 volumes ethanol. The mRNA was isolated using the OLIGOTEX kit (Qiagen, Chatsworth Calif.) and used to construct the cDNA library.

The mRNA was handled according to the recommended protocols in the SUPERSCRIPT plasmid system (Invitrogen). The cDNAs were fractionated on a SEPHAROSE CL4B column (APB), and those cDNAs exceeding 400 bp were ligated into pINCY plasmid (Incyte Genomics). The plasmid was subsequently transformed into DH5α competent cells (Invitrogen).

II. Isolation of cDNA Clones

Plasmid DNA was released from the cells and purified using the REAL Prep 96 plasmid kit (Qiagen). The recommended protocol was employed except for the following changes: 1) the bacteria were cultured in 1 ml of sterile TERRIFIC BROTH (BD Biosciences, San Jose Calif.) with carbenicillin at 25 mg/L and glycerol at 0.4%; 2) the cultures were incubated for 19 hours, and the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

III. Extension of CPCI Encoding Polynucleotides

The full length sequence of a cDNA having the nucleic acid sequence of SEQ ID NO:2 is produced by extension of an appropriate fragment of the full length molecule using oligonucleotide primers designed from this fragment. One primer is synthesized to initiate 5′ extension of the known fragment, and the other primer, to initiate 3′ extension of the known fragment. The initial primers are designed using OLIGO software (Molecular Insights, Cascade Colo.), or another program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations is avoided.

Selected human cDNA libraries were used to extend the sequence. If more than one extension is desired, additional or nested sets of primers are designed.

High fidelity amplification is obtained by PCR using methods well known in the art. PCR is performed in 96-well plates using the DNA ENGINE thermal cycler (M J Research). The reaction mix contains DNA template, 200 nmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B: 1: 94° C., 3 min; 2: 94° C., 15 sec; 3: 60° C., 1 min; 4: 68° C., 2 min; 5: 2, 3, and 4 repeated 20 times; 6: 68° C., 5 min; and 7: storage at 4° C. In the alternative, the parameters for primer pair T7 and SK+ are as follows: 1: 94° C., 3 min; 2: 94° C., 15 sec; 3: 57° C., 1 min; 4: 68° C., 2 min; 5: 2, 3, and 4 repeated 20 times; 6: 68° C., 5 min; and 7: storage at 4° C.

The concentration of DNA in each well is determined by dispensing 100 μl PICO GREEN quantitation reagent (0.25% (v/v) PICO GREEN) (Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate is scanned in a Fluoroskan II (Labsystems Oy, Helsinki, Finland) to measure the fluorescence of the sample and to quantify the concentration of DNA. A 5 μl to 10 μl aliquot of the reaction mixture is analyzed by electrophoresis on a 1% agarose mini-gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (APB). For shotgun sequencing, the digested nucleotides are separated on low concentration (0.6 to 0.8%) agarose gels, fragments are excised, and the agar is digested with AgarACE (Promega). Extended clones are religated using T4 ligase (New England Biolabs, Beverly, Mass.) into pUC 18 vector (APB), treated with Pfu DNA polymerase (Stratagene) to fill-in restriction site overhangs, and transfected into competent E. coli cells. Transformed cells are selected on antibiotic-containing media, and individual colonies are picked and cultured overnight at 37° C. in 384-well plates in LB/2× carb liquid media.

The cells are lysed, and DNA is amplified by PCR using Taq DNA polymerase (APB) and Pfu DNA polymerase (Stratagene) with the following parameters: 1: 94° C., 3 min; 2: 94° C., 15 sec; 3: 60° C., 1 min; 4: 72° C., 2 min; 5: 2, 3, and 4 repeated 29 times; S 6: 72° C., 5 min; and 7: storage at 4° C. The DNA is quantified by PICOGREEN reagent (Molecular Probes) as described above. Samples with low DNA recoveries are reamplified using the same conditions described above. Samples are diluted with 20% dimethysulphoxide (1:2, v/v), and sequenced using DYENAMIC energy transfer sequencing primers and the DYENAMIC DIRECT kit (APB) or the PRISM BIGDYE Terminator cycle sequencing ready reaction kit (ABI).

The nucleotide sequence of SEQ ID NO:2 may be used to obtain 5′ regulatory sequences using the procedure above, oligonucleotides designed for such extension, and an appropriate genomic library.

IV. Sequencing and Analysis

The cDNAs were prepared for sequencing using the CATALYST 800 (ABI) or the HYDRA microdispenser (Robbins Scientific, Sunnyvale Calif.) or MICROLAB 2200 (Hamilton) systems in combination with the DNA ENGINE thermal cyclers (M J Research). The cDNAs were sequenced using the PRISM 373 or 377 sequencing systems (ABI) and standard ABI protocols, base calling software, and kits. In one alternative, cDNAs were sequenced using the MEGABACE 1000 DNA sequencing system (APB). In another alternative, the cDNAs were amplified and sequenced using the PRISM BIGDYE Terminator cycle sequencing ready reaction kit (ABI). In yet another alternative, cDNAs were sequenced using solutions and dyes from APB. Reading frames for the ESTs were determined using standard methods (reviewed in Ausubel, 1997, supra, unit 7.7). Some of the cDNA sequences were selected for extension using the techniques disclosed in Example V.

The polynucleotide sequences derived from cDNA, extension, and shotgun sequencing were assembled and analyzed using a combination of software programs which utilize algorithms well known to those skilled in the art. Table 1 summarizes the software programs, descriptions, references, and threshold parameters used. The first column of Table 1 shows the tools, programs, and algorithms used, the second column provides a brief description thereof, the third column presents the references which are incorporated by reference herein, and the fourth column presents, where applicable, the scores, probability values, and other parameters used to evaluate the strength of a match between two sequences (the higher the probability the greater the homology). Sequences were further analyzed using MACDNASIS PRO software (Hitachi Software Engineering) and LASERGENE software (DNASTAR).

The cDNA sequences were validated by removing vector, linker, and polyA sequences and by masking ambiguous bases, using algorithms and programs based on BLAST, dynamic programming, and dinucleotide nearest neighbor analysis. The sequences were then queried against a selection of public databases such as GenBank primate, rodent, mammalian, vertebrate, and eukaryote databases, and BLOCKS to acquire annotation, using programs based on BLAST, FASTA, and BLIMPS. The sequences were assembled into full length cDNAs using programs based on Phred, Phrap, and Consed, and were screened for open reading frames using programs based on GeneMark, BLAST, and FASTA. The full length nucleic acid sequences were translated to derive the corresponding full length amino acid sequences, and these full length sequences were subsequently analyzed by querying against databases such as the GenBank databases (described above), SwissProt, BLOCKS, PRINTS, PFAM, and Prosite.

V. Northern Analysis and Transcript Imaging

Northern Analysis

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7 and Ausubel, supra, ch. 4 and 16.)

Analogous computer techniques applying BLAST are used to search for identical or related molecules in nucleotide databases such as GenBank or the LIFESEQ database (Incyte Genomics). This analysis is faster than multiple membrane-based hybridizations. In addition, the sensitivity of the computer search can be modified to determine whether any particular match is categorized as exact or homologous. The basis of the search is the product score, which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1% to 2% error, and, with a product score of 70, the match will be exact. Homologous molecules are usually identified by selecting those which show product scores between 15 and 40, although lower scores may identify related molecules.

The results of northern analysis are reported as a list of libraries in which the transcript encoding CPCI occurs. Abundance and percent abundance are also reported. Abundance directly reflects the number of times a particular transcript is represented in a cDNA library, and percent abundance is abundance divided by the total number of sequences examined in the cDNA library.

Transcript Imaging

A transcript image was performed using the LIFESEQ GOLD database (Incyte Genomics). This process allowed assessment of the relative abundance of the expressed polynucleotides in all of the cDNA libraries and was described in U.S. Pat. No. 5,840,484, incorporated herein by reference. All sequences and cDNA libraries in the LIFESEQ database are categorized by system, organ/tissue and cell type. The categories include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. Criteria for transcript imaging are selected from category, number of cDNAs per library, library description, disease indication, clinical relevance of sample, and the like.

For each category, the number of libraries in which the sequence was expressed are counted and shown over the total number of libraries in that category. For each library, the number of cDNAs are counted and shown over the total number of cDNAs in that library. In some transcript images, all enriched, normalized or subtracted libraries, which have had high copy number sequences removed prior to sequencing, and all mixed or pooled tissues, which may be considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs (such as heart, kidney, nerves or pancreas) would be aided by removing clinical samples from the analysis. Transcript imaging can also be used to support data from other methodologies such as hybridization, guilt-by-association and array technologies.

The transcript images for SEQ ID NOs:1 and 2 are shown below. The first column shows library name; the second column, the number of cDNAs sequenced in that library; the third column, the description of the library; the fourth column, absolute abundance of the transcript in the library; and the fifth column, percentage abundance of the transcript in the library.

SEQ ID NOs: 1 and 2
Category: Respiratory System (Lung)

| | cDNA | Description | | |
|---|---|---|---|---|
| Library* | s | of Tissue | Abundance | % Abundance |
| LUNGTUT09 | 3965 | lung tumor, | 2 | 0.0504 |
| | | squamous cell | | |
| | | CA, 68M | | |
| LUNLTUT04 | 2809 | lung tumor, | 1 | 0.0356 |
| | | squamous cell | | |
| | | CA, 65F | | |
| LUNGNOT23 | 11482 | lung, mw/mets | 2 | 0.0174 |
| | | osteoSAR, aw/ | | |
| | | pleura mets, | | |
| | | 58M | | |
| LUNGTUP07 | 30384 | lung tumor, | 1 | 0.0033 |
| | | neuroendocrine | | |
| | | carcinoid, pool, | | |
| | | SUB | | |

*cell lines removed, libraries with less than 2500 cDNAs not considered

SEQ ID NOs:1 and 2 were differentially expressed more than two-fold in squamous cell carcinoma of the lung when compared to expression in cytologically normal LUNGNOT23 and neuroendocrine carcinoid LUNGTUP07. SEQ ID NOs:1 and 2 were not differentially expressed in asthmatic lung tissue (LUNGAST01, LUNGNOT33, LUNGNOT38, and LUNGNOT39), idiopathic pulmonary disease (LUNGDIN02, LUNGDIS03), fetal lung (LUNGFEC01, LUNGFEM01, LUNGFER04, LUNGFET03, LUNGFET04, LUNGFET05, LUNGNOT09, LUNGNOT10, and LUNGNOT30), other normal lungs (LUNGNOE02, LUNGNOM01, LUNGNON03, LUNGNON07, LUNGNOP03, LUNGNOP04, LUNGNOT01, LUNGNOT02, LUNGNOT03, LUNGNOT05, LUNGNOT12, LUNGNOT14, LUNGNOT15, LUNGNOT18, LUNGNOT20, LUNGNOT22, LUNGNOT25, LUNGNOT27, LUNGNOT28, LUNGNOT31, LUNGNOT34, LUNGNOT35, and LUNGNOT37), or in any adenocarcinoma (LUNGTUP09, LUNGTUP15, LUNGTUT02, LUNGTUT06, LUNGTUT08, LUNGTUT10, LUNGTUT12, LUNGTUT13, LUNGTUT17, LUNLTMT01, and LUNPTUT02). SEQ ID NO:1, when used in a lung-specific assay is diagnostic for squamous cell carcinoma.

VI Hybridization and Amplication Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2× SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 110C oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45μl TE buffer, denaturing by heating to 100C for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for QPCR

Probes for the QPCR were prepared according to the ABI protocol.

Probe Preparation for Polymer Coated Slide Hybridization

The following method was used for the preparation of probes for the microarray analysis presented in FIG. 3. Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800× g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester N.Y.) is exposed to the membrane overnight at −70C, developed, and examined visually.

Polymer Coated Slide-Based Hybridization

The following method was used in the microarray analysis presented in Table 3. Probe is heated to 65C for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5× SSC in a corner of the chamber. The chamber containing the arrays is incubated for about 6.5 hr at 60C. The arrays are washed for 10 min at 45C in 1× SSC, 0.1% SDS, and three times for 10 min each at 45C in 0.1× SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of CyS. The excitation laser light is focused on the array using a 20× microscope objective (Nikon, Melville N.Y.). The slide containing the array is placed on a computer-controled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

Experiment and Results

The human promonocyte line, THP-1, was derived from peripheral blood of a 1-year-old Caucasian male with acute monocytic leukemia. THP-1 cells can be differentiated to the macrophage-like phenotype (which plays a critical role in the initiation and maintenance of inflammatory immune responses) by treatment with phorbol ester (PMA) for 24 hours. After activation, the macrophages were treated with oxLDL for three days and LPS (lipopolysacharride) for six hours.

Differences in gene transcription between monocytes and activated macrophages in response to lipopolysaccharide (LPS) were assessed. It was found that NFK activation and IL-12 production were affected and that CPCI showed more than a two-fold difference in expression where a two-fold difference was considered to be significant. The table below shows the log2 value of CPCI in this experiment using the Human Genome GEM 1 microarray.

| log2(Cy5/Cy3) | Sample (Cy3) | Sample (Cy5) |
|---|---|---|
| 1.086415 | THP1 t/PMA + oxLDL + LPS | THP1, t/PMA + oxLDL |

QPCR Analysis

For QPCR, cDNA is synthesized from 1 ug total RNA in a 25 ul reaction with 100 units M-MLV reverse transcriptase (Ambion, Austin Tex.), 0.5 mM dNTPs (Epicentre, Madison Wis.), and 40 ng/ml random hexamers (Fisher Scientific, Chicago Ill.). Reactions are incubated at 25C for 10 minutes, 42C for 50 minutes, and 70C for 15 minutes, diluted to 500 ul, and stored at −30C. Alternatively, cDNA is obtained from Human MTC panels (Clontech). PCR primers and probes (5′ 6-FAM-labeled, 3′ TAMRA) are designed using PRIMER EXPRESS 1.5 software (ABI) and synthesized by Biosearch Technologies (Novato Calif.) or ABI.

QPCR reactions are performed using an PRISM 7700 detection system (ABI) in 25 ul total volume with 5 ul cDNA template, 1× TAQMAN UNIVERSAL PCR master mix (ABI), 100 nM each PCR primer, 200 nM probe, and 1× VIC-labeled beta-2-microglobulin endogenous control (ABI). Reactions are incubated at 50C for 2 minutes, 95C for 10 minutes, followed by 40 cycles of incubation at 95C for 15 seconds and 60C for 1 minute. Emissions are measured once every cycle, and results are analyzed using SEQUENCE DETECTOR 1.7 software (ABI). Fold differences, relative concentration of mRNA as compared to standards, are calculated using the comparative C_{T} method (ABI User Bulletin #2).

VII Complementary Molecules

Molecules complementary to the cDNA, from about 5 (peptide nucleic acid) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in triple helix base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

VIII Expression of CPCI

Expression and purification of CPCI is achieved using bacterial or virus-based expression systems. For expression of CPCI in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express CPCI upon induction with isopropyl beta-D-thiogalactopyranoside (IPTG). Expression of CPCI in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding CPCI by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard et al. (1994) Proc Natl Acad Sci 91:3224-3227; Sandig et al. (1996) Hum Gene Ther 7:1937-1945.)

In most expression systems, CPCI is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma iaponicum, enables the purification of fusion proteins on immobilized glutathione under conditions that maintain protein activity and antigenicity (APB). Following purification, the GST moiety can be proteolytically cleaved from CPCI at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (Qiagen). Methods for protein expression and purification are discussed in Ausubel (supra, unit 16). Purified CPCI obtained by these methods can be used directly in the following activity assay.

IX. Demonstration of CPCI Activity

Peptidyl prolyl cis/trans isomerase activity of CPCI can be assayed by an enzyme assay described by Rahfeld et al. (1994; FEBS Lett 352:180-184). The assay is performed at 10° C. in 35 mM HEPES buffer, pH 7.8, containing chymotrypsin (0.5 mg/ml) and CPCI at a variety of concentrations. Under these assay conditions, the substrate, Suc-Ala-Xaa-Pro-Phe-4-NA, is in equilibrium with respect to the prolyl bond, with 80-95% in trans and 5-20% in cis conformation. An aliquot (2 ul) of the substrate dissolved in dimethyl sulfoxide (10 mg/ml) is added to the reaction mixture described above. The trans to cis conversion is measured by the hydrolysis of the cis conformer by chymotrypsin, producing 4-nitroanilide which is detected spectrophotometrically by absorbance at 390 nm. 4-nitroanilide appears in a time-dependent manner and is proportional to the amount of CPCI in the assay.

X. Functional Assays

CPCI function is assessed by expressing the sequences encoding CPCI at physiologically elevated levels in mammalian cell culture systems. cDNA is subcloned into a mammalian expression vector containing a strong promoter that drives high levels of cDNA expression. Vectors of choice include pCMV SPORT and pCR3.1 (Invitrogen), both of which contain the cytomegalovirus promoter. 5-10 μg of recombinant vector are transiently transfected into a human cell line of endothelial or hematopoietic origin using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., GFP (Clontech), CD64, or a CD64-GFP fusion protein. Flow cytometry (FCM), an automated, laser optics-based technique, is used to identify transfected cells expressing GFP or CD64-GFP, and to evaluate cellular properties, for example, their apoptotic state. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod (1994) Flow Cytometry, Oxford, New York N.Y.

The influence of CPCI on gene expression can be assessed using highly purified populations of cells transfected with sequences encoding CPCI and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding CPCI and other genes of interest can be analyzed by northern analysis or microarray techniques.

XI. Production of CPCI Specific Antibodies

CPCI purified using polyacrylamide gel electrophoresis (Harrington (1990) Methods Enzymol 182:488-495), or other purification techniques, is used to immunize rabbits and to produce antibodies using standard protocols.

Alternatively, the CPCI amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See Ausubel, supra, Unit 11.7.)

Typically, oligopeptides 15 residues in length are synthesized using an ABI 431A Peptide synthesizer (ABI) using fmoc-chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG.

XII. Purification of Naturally Occurring CPCI Using Specific Antibodies

Naturally occurring or recombinant CPCI is purified by immunoaffinity chromatography using antibodies specific for CPCI. An immunoaffinity column is constructed by covalently coupling anti-CPCI antibody to an activated chromatographic resin, such as CNBr-activated SEPHAROSE (APB). After the coupling, the resin is blocked and washed according to the manufacturer's instructions.

Media containing CPCI are passed over the immunoaffinity column, and the column is washed under conditions that allow the preferential absorbance of CPCI (e.g., high ionic strength buffers in the presence of detergent). The column is eluted under conditions that disrupt antibody/CPCI binding (e.g., a buffer of pH 2 to pH 3, or a high concentration of a chaotrope, such as urea or thiocyanate ion), and CPCI is collected.

XIII. Identification or Screening of Molecules Which Interact with CPCI

CPCI, or biologically active fragments thereof, are labeled with ¹²⁵I Bolton-Hunter reagent (Bolton et al. (1973) Biochem J 133:529-539). Candidate molecules previously arrayed in the wells of a multi-well plate are incubated with the labeled CPCI, washed, and any wells with labeled CPCI complex are assayed. Data obtained using different concentrations of CPCI are used to calculate values for the number, affinity, and association of CPCI with the candidate molecules.

In another alternative, libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIV Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra)

All patents and publications mentioned in the specification are incorporated by reference herein. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| | | | Parameter |
|---|---|---|---|
| Program | Description | Reference | Threshold |
| ABI FACTURA | A program that removes | Perkin- | |
| | vector sequences and | Elmer | |
| | masks ambiguous bases | Applied | |
| | in nucleic acid sequences. | Bio- | |
| | | systems, | |
| | | Foster | |
| | | City, CA. | |
| ABI/PARACEL | A Fast Data Finder useful | Perkin- | Mis- |
| FDF | in comparing and annotating | Elmer | match |
| | amino acid or nucleic acid | Applied | <50% |
| | sequences. | Bio- | |
| | | systems, | |
| | | Foster | |
| | | City, CA. | |
| | | Paracel | |
| | | Inc., | |
| | | Pasadena, | |
| | | CA. | |
| ABI | A program that assembles | Perkin- | |
| AutoAssembler | nucleic acid sequences. | Elmer | |
| | | Applied | |
| | | Bio- | |
| | | systems, | |
| | | Foster | |
| | | City, CA. | |
| BLAST | A Basic Local Alignment | Altschul, | ESTs: |
| | Search Tool useful in | S. F. et al. | Prob- |
| | sequence similarity search | (1990) J. | ability |
| | for amino acid and nucleic | Mol. Biol. | value = |
| | acid sequences. BLAST | 215:403- | 1.0E-8 |
| | includes five functions: | 410; | or less |
| | blastp, blastn, blastx, | Altschul, | Full |
| | tblastn, and tblastx. | S. F. et al. | Length |
| | | (1997) | sequences: |
| | | Nucleic | Prob- |
| | | Acids | ability |
| | | Res. 25: | value = |
| | | 3389- | 1.0E- |
| | | 3402. | 10 or less |
| FASTA | A Pearson and Lipman | Pearson, | ESTs: |
| | algorithm that searches | W. R. and | fasta E |
| | for similarity between a | D. J. Lip- | value = |
| | query sequence and a group | man | 1.06E-6 |
| | of sequences of the same | (1988) | Assem- |
| | type, FASTA comprises at | Proc. Natl. | bled |
| | least five functions: | Acad Sci. | ESTs: |
| | fasta, tfasta, fastx, | 85:2444- | fasta |
| | tfastx, and search. | 2448; | Identity = |
| | | Pearson, | 95% or |
| | | W. R. | greater |
| | | (1990) | and Match |
| | | Methods | length = |
| | | Enzymol. | 200 bases |
| | | 183:63- | or greater; |
| | | 98; and | fastx |
| | | Smith, T. | E value = |
| | | F. and M. | 1.0E-8 |
| | | S. Water- | or less |
| | | man | Full |
| | | (1981) | Length |
| | | Adv. | sequences: |
| | | Appl. | fastx |
| | | Math. 2: | score = |
| | | 482-489. | 100 or |
| | | | greater |
| BLIMPS | A BLocks IMProved | Henikoff, | Score = |
| | Searcher that matches a | S and J. | 1000 or |
| | sequence against those | G. Heni- | greater; |
| | in BLOCKS and PRINTS | koff, | Ratio of |
| | databases to search for | Nucl. | Score/ |
| | gene families, sequence | Acid | Strength = |
| | homology, and structural | Res., | 0.75 or |
| | fingerprint regions. | 19:6565- | larger; |
| | | 72, 1991. | and |
| | | J. G. | Prob- |
| | | Henikoff | ability |
| | | and S. | value = |
| | | Henikoff | 1.0E- |
| | | (1996) | 3 or less |
| | | Methods | |
| | | Enzymol. | |
| | | 266.88- | |
| | | 105; | |
| | | and | |
| | | Attwood, | |
| | | T. K. et | |
| | | al. (1997) | |
| | | J. Chem. | |
| | | Int. | |
| | | Comput. | |
| | | Sci. | |
| | | 37:417- | |
| | | 424. | |
| PFAM | A Hidden Markov Models- | Krogh, A. | Score = |
| | based application useful | et al. | 10-50 |
| | for protein family search. | (1994) | bits, |
| | | J. Mol. | depend- |
| | | Biol., | ing on |
| | | 235:1501- | indi- |
| | | 1531; | vidual |
| | | Sonn- | protein |
| | | hammer, | families |
| | | E. L. L. et | |
| | | al. (1988) | |
| | | Nucleic | |
| | | Acids Res. | |
| | | 26:320- | |
| | | 322. | |
| ProfileScan | An algorithm that searches | Gribskov, | Score = |
| | for structural and sequence | M. et al. | 4.0 or |
| | motifs in protein sequences | (1988) | greater |
| | that match sequence patterns | CABIOS | |
| | defined in Prosite. | 4:61-66; | |
| | | Gribskov, | |
| | | et al. | |
| | | (1989) | |
| | | Methods | |
| | | Enzymol. | |
| | | 183:146- | |
| | | 159; | |
| | | Bairoch, | |
| | | A. et al. | |
| | | (1997) | |
| | | Nucleic | |
| | | Acids Res. | |
| | | 25:217- | |
| | | 221. | |
| Phred | A base-calling algorithm | Ewing, B. | |
| | that examines automated | et al. | |
| | sequencer traces with high | (1998) | |
| | sensitivity and probability. | Genome | |
| | | Res. 8: | |
| | | 175-185; | |
| | | Ewing, B. | |
| | | and P | |
| | | Green | |
| | | (1998) | |
| | | Genome | |
| | | Res. 8: | |
| | | 186-194. | |
| Phrap | A Phils Revised Assembly | Smith, | Score = |
| | Program including SWAT and | T. F. | 120 or |
| | CrossMatch, programs based | and M. S. | greater, |
| | on efficient implementation | Waterman | Match |
| | of the Smith-Waterman | (1981) | length = |
| | algorithm, useful in | Adv. | 56 or |
| | searching sequence homology | Appl. | greater |
| | and assembling DNA | Math. | |
| | sequences. | 2:482- | |
| | | 489; | |
| | | Smith, T. | |
| | | F. and M. | |
| | | S. Water- | |
| | | man | |
| | | (1981) J. | |
| | | Mol. Biol. | |
| | | 147:195- | |
| | | 197; and | |
| | | Green, P., | |
| | | Univer- | |
| | | sity of | |
| | | Washing- | |
| | | ton, | |
| | | Seattle, | |
| | | WA. | |
| Consed | A graphical tool for viewing | Gordon, | |
| | and editing Phrap assemblies | D. et al. | |
| | | (1998) | |
| | | Genome | |
| | | Res. 8: | |
| | | 195-202. | |
| SPScan | A weight matrix analysis | Nielson, | Score = |
| | program that scans protein | H. et al. | 5 or |
| | sequences for the presence | (1997) | greater |
| | of secretory signal peptides. | Protein | |
| | | Engineer- | |
| | | ing 10: | |
| | | 1-6; | |
| | | Claverie, | |
| | | J. M. and | |
| | | S. Audic | |
| | | (1997) | |
| | | CABIOS | |
| | | 12:431- | |
| | | 439. | |
| Motifs | A program that searches | Bairoch | |
| | amino acid sequences for | et al. | |
| | patterns that matched | supra; | |
| | those defined | Wisconsin | |
| | in Prosite. | Package | |
| | | Program | |
| | | Manual, | |
| | | version | |
| | | 9, page | |
| | | M51-59, | |
| | | Genetics | |
| | | Computer | |
| | | Group, | |
| | | Madison, | |
| | | WI. | |

What is claimed is:

1. A purified protein comprising the amino acid sequence of SEQ ID NO:1 or a fragment of SEQ ID NO:1.

2. A purified antibody that specifically binds to the protein of claim 1.

3. The antibody of claim 2, wherein the antibody is selected from an intact immunoglobulin molecule, a polyclonal antibody, a monoclonal antibody, a chimeric antibody, a recombinant antibody, a bispecific antibody, a multispecific antibody, a humanized antibody, a single chain antibody, a Fab fragment, an F(ab′)₂ fragment, an Fv fragment; an agonist antibody, and an antibody-peptide fusion protein.

4. A method of using a protein to prepare and purify a polyclonal antibody comprising:
a) immunizing a animal with a protein having the amino acid sequence of SEQ ID NO:1 or a fragment of SEQ ID NO:1 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

5. A method of using a protein to prepare a monoclonal antibody comprising:
a) immunizing a animal with a protein having the amino acid sequence of SEQ ID NO:1 or a fragment of SEQ ID NO:1 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies from culture.

6. A method for using an antibody to immunopurify a protein comprising:
a) attaching the antibody of claim 2 to a substrate,
b) exposing the antibody to a sample containing protein under conditions to allow antibody:protein complexes to form,
c) dissociating the protein from the complex, and
d) collecting the purified protein.

7. A polyclonal antibody produced by the method of claim 4.

8. A monoclonal antibody produced by the method of claim 5.

9. An method for using an antibody to detect expression of a protein in a sample, the method comprising:
a) combining the antibody of claim 2 with a sample under conditions which allow the formation of antibody:protein complexes; and
b) detecting complex formation, wherein complex formation indicates expression of the protein in the sample.

10. The method of claim 9 wherein the method is selected from two-dimensional polyacrylamide gel electrophoresis, western analysis, mass spectrophotometry, enzyme-linked immunosorbent assays, radioimmunoassays, fluorescence-activated cell sorting, protein arrays, and antibody arrays.

11. The method of claim 9 wherein complex formation is compared with standards and is diagnostic of squamous cell carcinoma or atherosclerosis.

12. A composition comprising an antibody of claim 2 and a labeling moiety.

13. A composition comprising an antibody of claim 2 and a pharmaceutical agent.

14. A kit comprising the composition of claim 12.

15. An array comprising the composition of claim 12.

16. A method for delivering a pharmaceutical agent to treat a neoplastic disorder or immune response associated with expression of a protein having the amino acid sequence of SEQ ID NO:1, the method comprising administering to a subject in need of such treatment a bispecific antibody of claim 3, wherein the antibody specifically binds the protein having the amino acid sequence of SEQ ID NO:1 and the pharmaceutical agent.

17. A method for treating a disorder associated with expression of a protein having the amino acid sequence of SEQ ID NO:1, the method comprising administering to a subject in need of such treatment the antibody of claim 2.

18. A purified agonist that specifically binds the protein of claim 1.

19. A purified antagonist that specifically binds the protein of claim 1.

20. A method for treating a disorder associated with differential expression or activity of a protein having the amino acid sequence of SEQ ID NO:1, the method comprising administering to a subject in need of such treatment an effective amount of the composition of claim 13.


SEQUENCE LISTING (12 sequences)

SEQ ID NO: 1 - protein, 161 aa, Homo sapiens
  misc_feature: Incyte ID No: 2925455CD1
MSVTLHTDVGDIKIEVFCERTPKTCENFLALCASNYYNGCIFHRNIKGFMVQTGDPTGTGRGGNSIWGKKFEDEYSEYLKHNVRGVVSMANNGPNTNGSQFFITYGKQPHLDMKYTVFGKVIDGLETLDELEKLPVNEKTYRPLNDVHIKDITIHANPFAQ

SEQ ID NO: 2 - DNA, 1069 bp, Homo sapiens
  misc_feature: Incyte ID No: 2925455CB1
cgcgcccaacttccgctggcccaaagaaactataattttgaaccaacagacctctgctggcatctgcgattgcatttttcctgttttaacaacggctgtgctagacgaagtggtgaagcccaaagacttatttttgagctcgctgtaagactgagaaatcacgtagtccttcctgaaaccactaagaggaaaaatgtctgtgacactgcatacagatgtaggtgatattaaaattgaagtcttctgtgagaggacacccaaaacatgtgagaatttcttggctctttgtgccagtaattactacaatggctgtatatttcataggaatatcaagggtttcatggttcaaacaggagatccaacaggaactggaagaggaggcaacagtatttggggcaagaagtttgaggatgaatacagtgaatatcttaagcacaatgttagaggtgttgtatctatggctaataatggcccgaacaccaatggatctcagttcttcatcacctatggcaaacagccacatttggacatgaaatacaccgtatttggaaaggtaatagatggtctggaaactctagatgagttggagaagttgccagtaaatgagaagacataccgacctcttaatgatgtacacattaaggacataactattcatgccaacccatttgctcagtagctatgatagacctggacaaataacttgacaaattgctggaacacacttattgtggtttacccggttttaattatgtcagagattgcatcatccttctgcttgtttacaactatgatcttctatgaaatggtggtaccaaggggcgcccaacagcttttatccccattcttagagcatattctttattataatgattatccaacatatttctttaattttaatacaaaaaatacatcatttaatttttgttacatatgaacattcatttttaaatgctcagcctcaagtgcaggcatttttgagtggcctgattacatattcctcccacagcaagtccgtatcctggaagtgttattttataataaaatttaaaaagttttaaaaaaaaaa

SEQ ID NO: 3 - DNA, 248 bp, Homo sapiens
  misc_feature: Incyte ID No: 2925455H1
atttcataggaatatcaagggtttcatggttcaaacaggagatgcaacaggaactggaagaggaggcaacagtatttggggcaagaagtttgaggatgaatacagtgaatatcttaagcacaatgttagaggtgttgtatctatggctaataatggcccgaacaccaatggatgtcagttcttcatcacctatggcaaacagccacatttggacatgagatacaccgtatttggaaaggtaatagatg

SEQ ID NO: 4 - DNA, 480 bp, Homo sapiens
  misc_feature: Incyte ID No: 2619977R6
  unsure 2, 452: a, t, c, g, or other
cncgcccaacttccgctggcccaaagaaactataattttgaaccaacagacctctgctggcatctgcgattgcatttttcctgttttaacaacggctgtgctagacgaagtggtgaagcccaaagacttatttttgagctcgctgtaagactgagaaatcacgtagtccttcctgaaaccactaagaggaaaaatgtctgtgacactgcatacagatgtaggtgatattaaaattgaagtcttctgtgagaggacacccaaaacatgtgagaatttcttggctctttgtgccagtaattactacaatggctgtatatttcataggaatatcaagggtttcatggttcaaacaggagatccaacaggaactggaagaggaggcaacagtatttggggcagaagtttgaggatgaatacagtgaatatcttaagcacaatgttagaggtgttgnatctatgggctaataatgggcccgaaca

SEQ ID NO: 5 - DNA, 564 bp, Homo sapiens
  misc_feature: Incyte ID No: 586886F1
aaaactttttaaattttattataaaataacacttccaggatacggacttgctgtgggaggaatatgtaatcaggccactcaaaaatgcctgcacttgaggctgagcatttaaaaatgaatgttcatatgtaacaaaaattaaatgatgtattttttgtattaaaattaaagaaatatgttggataatcattataataaagaatatgctctaagaatggggataaaagctgttgggcgccccttggtaccaccatttcatagaagatcatagttgtaaacaagcagaaggatgatgcaatctctgacataattaaaaccgggtaaaccacaataagtgtgttccagcaatttgtcaagttatttgtccaggtctatcatagctactgagcaaatgggttggcatgaatagttatgtccttaatgtgtacatcattaagaggtcggtatgtcttctcatttactggcaacttctccaactcatctagagtttccagaccatctattacctttccaaatacggtgtatttcatgtcccaaatgtgggctgtttgccataggtgatga

SEQ ID NO: 6 - DNA, 1032 bp, Rattus norvegicus
  misc_feature: Incyte ID No: 222746_Rn.1
gaggtatctgggcgctgtaaggttgtccgtagaaccctctcgcggtcagagggggccgaggagtgaggtgcatgccccacctccccattccacccctccccaggagccctagtccgtgtgctgaggagagactggcgggagagtagggcgttctctgcggagagccccgcgcctcacttccggtggtgcattgacactcttggaacccgcaggcatgccggcacctgtgaatgagacatttttgttaccgcagcagcggtgctgagcgaggagctttgaaaaattaaatagtgctccctagaagcatcaagaaggagaatgtctgtgacactgcatacagatgtgggagatatcaagatagaagtcttctgtgagagaacacccaaaacctgtgagaatttcttggctctgtgtgccagtaattactacaatggctgtgtgttccatagaaatatcaagggcttcatggttcaaacaggagatccaacaggtactggaagaggaggcagcagtatctggggcaagaagtttgaggatgaatacagtgaatatctgaagcacaatgttcgaggtgttgtatctatggctaataatggcccaaacaccaacggatctcagttcttcatcacctatggcaagcagccacacttggacatgaaatatacagtgttcggaaaggtaatagatggtctggagactttggatgaattggagaagttaccagtaaatgagaagacttacagacctcttaatgatgtgcacattaaggacataactattcatgccaacccgtttgctcagtagctgtaataggcctggccacacactggcaaacacttaactgggaaacgcccagtgtggtttatccagttctgagtccatcagagactggtccttctgctggcatctacagtactgggctccatgcgggagccctggggttcacgccccatcttgaacatattctgtgccacggctgtcgctcaataggtttactttaaatagagagcagtgccttgtttttatcaaaaa

SEQ ID NO: 7 - DNA, 175 bp, Rattus norvegicus
  misc_feature: Incyte ID No: 252329_Rn.1
gccgagtgtaaagcattgcttgaggttgcccagagacttatttttgagctcgctgtaagactgagaaatcacgtagtccttcctgaaaccactaagaggaaaaatgtctgtgacactgcatacagatgtaggtgatattaaaattgaagtcttctgtgagaggacacccaaaaca

SEQ ID NO: 8 - DNA, 302 bp, Canis familiaris
  misc_feature: Incyte ID No: 023828_Cf.1
gatacacacccctaacattgtgctgtcggatctcctgtttgaaccatgaaacccttgatatttctatgaaatatacagccattgtagtaattactggcacaagagccaagaaattctcacaagttttgggtgtcctctcacagaatacttcaattttaatatcacctacatctgtatgcagtgtcactgacatttttcttcttgatgtgagtttcaggaagtactattgttcaatttctcagtctcaccaagagaagttagaaaacgtgtctctggtacgcttttgacagggagagggggct

SEQ ID NO: 9 - DNA, 1146 bp, Canis familiaris
  misc_feature: Incyte ID No: 024646_Cf.1
  unsure 670-833: a, t, c, g, or other
taccaaatctaatcaaggtgatggaggcaagatcaaagaatacggttttgatgagatcaatgatatatatctatatatttatagatatataaatgatgaggcctgattaggtgagaccttatggtggagataaatattaagagaatagcttctgtttactcactacagaacacttatcagatgccagaagctttacattgtttcattagtcctcacagtcttatgaaggggatgagtctttagggatgagaaaatctgggagatggtaacctgcctaaagttactagtaggaggtagagctgtgattcaaacacaatccaggactctacaactggtgctcttagccaattagttgcatttttcaaacagatcacctgtagaattctgtcctacaatactgtatatgatagctttaaaaaagttccatggaaaaaggaattgggaaattgagtgcttactctactctaggacttctctgagcctttactgtaatctaaagaccacagaaaatagcaaatggattggctggctggcttttctagatcatctctatcttgtccttactctgactgcacaaagtgaataattaatggaaaagactacctctaggacgtggactagatatggcaggaatatgcatagtgcttaaaaatgccaggatttgggcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaatgcctatatgtaaccaaaaattagaagagttttttggaaaattaaacttaaataagatcttagctataaacaagcagaaggatgatgtaatcttttgacataattaaaactgggtaaaccacaataggtgtgttccttaaatagtctgccaaatatttttctaggtctattatagctattgtgcaaatggattggcatgaatagttatatccttaatgtgtacgtcattaagaggccggtagtcttctcatttactggtaacttctccaattcatctagagtttctagaccatctattactttccaaaac

SEQ ID NO: 10 - DNA, 2922 bp, Mus musculus
  misc_feature: Incyte ID No: 023075_Mm.3
agagaaggtttgcctttaatgtactctttacagacagttttaccgaatgagctatggcttttgttgagccagagtcgtgaactgaaatgagaagtccaatacagctctgcagagggtggaacaccaactcctgcacagccgtttcaatccaggtgatctgtggcttctgtcatcatggtgtccttgacgtccttgcatttaaaccacacggagagggtccctgtctgtctcagacaggataatgttaatcttattctcaaagtgaacacccagcatttcttgaagctcagaaaggaagcctctttcagtgttgctgtgttcacaaaggatgacattgatccctttggaagcagcatccagaacatcatggtgggacatttcacctgtgaggtaaaggtcggcctccactccttgtagaacactgcccccagaaccagcacacagggccacaactttgacttgggactctaatgttctccccactccaagagctaagcgaagatgcgacagctttaggtgtgttttgattcgctctatcattattgccagggagacagattcatccagtgtgcacaaccgtcccattccagtatgcagaagcaaaggcttctccaatgaaagaatttcagttttctgataaagttgtctgtcctgggaaagaaaggctagcacctgcatcaaagtcttctgagtacaattcaggctgatccgggtttgctcttcaccatcacacctggcaggaaaagaagtgacagagacacctccaacccctctcagtgtagacatgactttgtccaggtcttggctgcggttcacactgaattctagtcggtgagctccctctgtggggtagtctggagctctggaagggtggatgggcctggtagtgcaagttccaagccctttggccaaccagctgttgactccctggggtgccgcatcataggctgtgtggggagagtagacagcgactctgttctccagagcccggatcacgagacactccttccaggttttccaagttatgtgcttcatgggccggaaaataggtggatggtaggagagaatgaagtctgccttcttttgcagagcctcgtccatgacctcctccgtcaggtcattggtcaggaagagtgtatttacagtatggggtgggcttggctccaccagtaatcccacattgtcccagctctcagcaaatgagagggatgcaaagtcattcaaggacgagagaagagccttcagatccatgaaggaacgggaagacctgcagatccacgactgggcacgttgcacacttgtagggactaggtgtgccgatgacagcatacggaaaccagggagaagggtgccagtcgcagaagctctttcagtgtatcacttccgctctgctctgaagctcctggctttgtggaaagagttgggcagaagtggtcctgtgcgttctgcttccgactccaggccgctgtgtgaagttgggcggaagttgtcctgtgcgttctgcttccgactccaggccgctgtgtgaagttgggcggaattggtcctgtgcgttctgcttcccactccaggccgctgtatgaagttgctgggcgacagcgggttctgcttcttagtgaggttgtgggtagagctctcaggggccgaggggtgaggtgcgtggtcctccgagtcctagtcggtgtgatgaggagagactgacgggagagtagggcgtcccctgcggagggctccgggcttcacttctgtggcgcacagacacgcctttggaacccgcaggcatgccggcccttgtagttgaggcttttccgttcccgcagcagtggtgctgagcgcagtgggtgagtgtgggggaaaggcttttatccttttctccgtgcactcaaacgcctgctgcgccccacgttggtgcaaatttatttcttgggatcctcgtggtgaactctcgggctggagatggtattcctgggacttttaaatacgattgataagttagcaagatgtggcgtttgggaaaaaacaaaacaaaagtgtgaagtatagaaatgtattgtacagacgtcttaagtaaagtaggtgtcccgggtaaagggagctccaagtgcctatctagagcactagacctggaggtggggctatgcctcccttgcagaaagccttgaagaaaatgagtaacagagattccgaaacaattaaacagtgcttcctagaaccatcaagaaggaaaatgtctgtgacactgcatacagatgtgggagatatcaagatagaagtcttctgtgagagaacacccaaaacatgtgagaatttcttggctctttgtgccagtaattactacaacggctgtgtgtttcatagaaatatcaagggcttcatggttcaaacaggagatccaacaggtactggaagaggaggtagcagcatctgggccaaaaagtttgaggatgaatacagtgaatatctgaagcacaatgttcgaggtgttgtatctatggctaataatggcccaaataccaatggatctcagttcttcatcacctatggcaagcagccacacttggacatgaaatatacagtgtttggaaaggtaatagatggtctggagactttggatgagttggagaagttaccagtaaatgagaagacatacagacctcttaatgatgtacacattaaggacataactattcatgccaacccatttgctcagtagctgtcatggacctggacagatacttggtgaacacttcactggaacacgcccattgtggtttaaccagttctccttctcctggtgtttgcaggaatgggctccatgtaggagccctgtagcttccctcccatctgagcatattctctgccactgctgttgttcagtgtgttcactttaaatagaaagccatgccttttttttttatcacg

SEQ ID NO: 11 - protein, 147 aa, Caenorhabditis elegans
  ref <308>: DATABASE ACCESSION NUMBER: Genbank ID No: g1155225
  ref <309>: DATABASE ENTRY DATE: 14 June 1996
MSVTLHTTSGDIKIELYVDDAPKACENFLALCASDYYNGCIFHRNIKDFMVQTGDPTHSGKGGESIWGGPFEDEFVSALKHDSRGCVSMANNGPDSNRSQFFITYAKQAHLDMKYTLFGKVIDGFDTLEEIETIKVDNKYRPLVQQK

SEQ ID NO: 12 - protein, 282 aa, Homo sapiens
  ref <308>: DATABASE ACCESSION NUMBER: Genbank ID No: g1199600
  ref <309>: DATABASE ENTRY DATE: 11-22-96
AHYSTGKVSASFTSTAMVPETTHEAAAIDEDVLRYQFVKKKGYVRLHTNKGDLNLELHCDLTPKTCENFIRLCKKHYYDGTIFHRSIRNFVIQGGDPTGTGTGGESYWGKPFKDEFRPNLSHTGRGILSMANSGPNSNRSQFFITFRSCAYLDKKHTIFGRVVGGFDVLTAMENVESDPKTDRPKEEIRIDATTVFVDPYEEADAQIAQERKTQLKVAPETKVKSSQPQAGSQGPQTFRQGVGKYINPAATKRAAEEEPSTSATVPMSKKKPSRGFGDFSSW